(19) United States
(12) Patent Application Publication
(10) Pub. No.: US 2026/0130840 A1
(43) Pub. Date: May 14, 2026

(54) COSMETIC COMPOSITION FOR COSMETIC PRODUCTS, CONTAINING A LINEAR OR BRANCHED POLYURETHANE WITH HIGH NATURAL ORIGIN INDEX (NOI)

(71) Applicant: INTERCOS S.p.A., MILANO (IT)

(72) Inventors: Nella GALOTTO GALOTTO, LOMAGNA (LC) (IT); Claudio PIROVANO, VERDERIO (LC) (IT); Gaetano DISTEFANO, BERGAMO (IT); Patrizia VALSESIA, CALCO (LC) (IT); Sara BETTINELLI, PARABIAGO (MI) (IT); Gabriele DEPTA, MONZA (IT)

(73) Assignee: INTERCOS S.p.A., MILANO (IT)

(21) Appl. No.: 19/106,839

(22) PCT Filed: Sep. 8, 2023
(86) PCT No.: PCT/EP2023/074791

§ 371 Date: Feb. 26, 2025

(30) Foreign Application Priority Data
Sep. 12, 2022 (IT) 102022000018588

(51) Int. Cl. A61K 8/87; A61Q 1/02

(52) U.S. Cl. CPC A61K 8/87; A61Q 1/02; A61K 2800/26; A61K 2800/95

(57) ABSTRACT

Cosmetic composition for cosmetic products with high naturalness, containing at least a linear or branched polyurethane with Natural Origin Index (NOI) higher than 0.85, comprising at least a polyester polyol with NOI=1, made totally of natural components or of components of natural origin, an aliphatic diisocyanate or a triisocyanate, further comprising at least one cosmetic oil with function of solvent for the synthesis of polyurethane. Said polyurethane is able to form a film on skin, and is preferably provided with gloss. (FIG. 1)

The present invention relates to a cosmetic composition for cosmetic products, containing one or more linear or branched polyurethanes with a high Natural Origin Index (NOI), obtained from polyols of natural origin, especially selected to obtain linear and branched structures, capable of forming a film on the skin, with distinct rheologic features and a good compatibility with the most common cosmetic ingredients. In their preferred embodiments, such polyurethanes are provided with a glossy finish.

In particular, the present invention relates to a formulation of cosmetic products (of any category) with high naturalness (Natural Origin Content>80%), comprising linear or branched polyurethanes of natural origin.

Cosmetic industry is moving at high speed toward sustainability goals, pushed not only by industry regulations and standard, (e.g. microplastics), but also by the concerns of society about the final destination of the chemical substances present in each formulation (e.g. silicones), the chemicals used during production (e.g. ethylene oxide used for PEG derivatives), ethical origin (palm oil and palm oil derivatives, mica, talc), the natural origin (naturalness) of the ingredients, the energy consumption for the production of ingredients and products, etc.

In order to satisfy such requirements, cosmetic manufacturers have to continuously modify their formulas, providing the removal of undesired ingredients and their replacement with sustainable, bio-based and biodegradable raw materials, ensuring the same cosmetic performances. Reformulations are a daily task for cosmetic formulators, but the continuous removal of undesired ingredients from their “artist's palette” proves challenging.

Two recently introduced parameters concerning the natural origin of cosmetic raw materials and of cosmetic products are the Natural Origin Index (NOI) or “naturalness index”, and the Natural Origin Content (NOC), according to ISO 16128-2:2017 standard “Guidelines on technical definitions and criteria for natural and organic cosmetic ingredients—Part 2: Criteria for ingredients and products”.

NOI is a value indicating the extent to which a cosmetic ingredient satisfies the definition of natural ingredient, derived natural ingredient or mineral derived ingredient indicated in the ISO 16128-1:2016 standard “Guidelines on technical definitions and criteria for natural and organic cosmetic ingredients and products-Part 1: Definitions for ingredients” Web URL: https://www.iso.org/standard/62503.html. NOI can take values>0.5 and ≤1, wherein 1 is the maximum degree of natural origin, while the ingredients with a calculated value of ≤0.5 have a NOI equal to 0.

The NOC of a product is the mass percentage, comprised between 0% and 100%, of all the natural ingredients, natural portions of ingredients and ingredients of natural origin in the product. It is calculated as the sum of the relative concentrations of the ingredients of a product, multiplied by their corresponding NOI.

These parameters represent a standard that today is widely used for positioning cosmetic ingredients and products in terms of naturalness, considering mainly the origin of the raw material and the process used for producing it. The demand for cosmetics with a high natural index is nowadays commonplace, indicating a higher and higher perception and awareness of consumers concerning the topic of sustainability in a broad sense.

In order to meet these continuous restrictions, the adoption of previously neglected ingredients and their re-evaluation as alternatives is of great value. Nonetheless, the process is long and poorly rationalizable, as the relationship between chemical structure and physical properties, as well as the relationship between physical properties and cosmetic properties, is difficult to be foreseen. For this reason, the choice of alternative ingredients often requires a long, iterative “trial and error” process.

Moreover, focusing on a specific category of cosmetic raw materials like polymers, ideating ingredients with a high NOI, while at the same time maintaining the performances of the common polymers having a petrochemical origin, is far from obvious. The features that cosmetic polymers can provide to finished products are the result of years of optimization of synthetic designing of the polymer structure, so as to obtain materials capable of forming more or less self-consistent films on the surface of skin, but especially providing long-lasting properties and resistance to water in all its forms (sea water, pool water, tears, sweat, etc.).

Polyurethanes are the preferred platform for the research of materials with customized film-forming and structuring properties, allowing the creation of chemical structures through the choice of co-monomers: diisocyanates with a stiff or flexible structure (isophorone diisocyanate, IPDI or hexamethylene diisocyanate, HDI), polyols and their relative quantity, and obviously the overall stoichiometry determining the final molecular weight [Galotto N, Pirovano C, Distefano G, Saligari F, Valsesia P, Bettinelli S, Depta G, (2019) “Shaping anhydrous cosmetic products by playing with polyurethanes chemistry”. 25th IFSCC Conference (Milan, Italy)]. The solvent chosen as synthesis means plays an important role, too. In fact, using a cosmetically accepted ingredient, that is a solvent compatible both with the starting monomers and with the finished product in order to ensure a stable blend, leads to a great added value.

Recent patents show the great interest for this topic, but none of them shows the achievement of cosmetic formulations containing polyurethanes with a high NOI with cosmetic properties analogues to those of cosmetic formulations containing formulations of petrochemical origin, concerning the ability to form films on the skin, particularly glossy films.

US 2021/059924 relates to the use of a bio-based and biodegradable elastomeric rubber polyurethane, formed by a polyol reticulated with a bio-based isocyanate, synthetized with a bismuth catalyst, in the presence of a cosmetic emollient. Its aim is providing alternatives to the cosmetic rubbers of fossil origin, in particular silicone rubbers, dispersed in different cosmetic fluids. The obtained rubber is then high shear milled in order to get particles of dimensions smaller than 100 microns. The obtained rubbers cannot be used as film-forming polymers. NOI is not considered as a driver for the ideation of such elastomers and the selection of reticulated polyols. Finally, the salient features required from cosmetic polyurethanes, i.e. hydrophobicity and the ability to form glossy and uniform films on the skin are not described in the document.

US 2020/109231 describes oil gelling polyurethanes producing transparent gels, and a process for their production. Said gelling polyurethanes are prepared with a three-step process: 1) functionalization of a di-OH estolide with a diisocyanate derivative in order to obtain a diisocyanate estolide; 2) extension of the chain by addition of a difunctional compound capable of reacting with the isocyanates of the compound, optionally solubilized in an oil; 3) optionally, chain termination by addition of a nucleophilic compound capable of reacting with optionally residual isocyanate functions. Said gelling polyurethanes are provided with a high NOI and are used as gelling agents for cosmetic products. No hint is given in the document about their film-forming properties, much less glossy films.

EP 3636321 describes aqueous polyurethane urea dispersions based on polyester polyols used as coating compositions. Said polyurethanes are used in cosmetic products for hair, nails or skin.

US 2016/272751 relates to a polyurethane derived from polyester-polyol-based biomass resources for manufacturing synthetic or artificial leathers, foamed resins for shoe sole, thermoplastic resins, thermosetting resins, paints, laminating adhesives and elastic fibers. The cosmetic use is not considered; in fact, the polyols are not selected for obtaining a polyurethane with high adherence to skin and all the other cosmetic properties required in a cosmetic ingredient.

Moreover, on the market there are provided polymers with a high NOI, but specifically sponsored for aqueous formulations, wherein the synthesis solvent is water: they cannot be used in anhydrous formulations not containing water.

Therefore, the present invention is connected to the ideation of new polyurethane polymers with a high NOI, starting from polyols of natural origin, specially selected to obtain linear and branched structures, capable of forming a more or less self-consistent film on the skin, with distinct rheologic features and a good compatibility with the most common cosmetic ingredients. In their preferred embodiment, such polyurethanes are provided with a glossy finish.

From prior research experiences [Galotto N, Pirovano C, Distefano G, Saligari F, Valsesia P, Bettinelli S, Depta G, (2019) “Shaping anhydrous cosmetic products by playing with polyurethanes chemistry”. 25th IFSCC Conference (Milan, Italy)], [Distefano G, Pirovano C, Mottadelli S, La Vardera M, Vitali A, Follis R, Valsesia P, Bettinelli S and Depta G (2015) “Glossy comb-like polyurethane film formers with optimized cosmetic properties”. 23rd IFSCC Conference (Zurich, Switzerland)], [Morlacchi S, Salanti A (2008) “Cosmetic composition comprising a polyurethane based on dialkyl tartrate diol and uses thereof, WO 2010/049480”] it was gathered that structures containing Di-C12-13 alkyl tartrate and hydrogenated dilinoleyl alcohol and HDI and/or IPDI show suitable film-forming properties, adherence to skin and resistance to water. Nonetheless, alkyl tartrate C12-13 as main monomeric diol has the drawback of having a full petrochemical origin. Moreover, the isododecane used as solvent is of petrochemical origin, as well as the diisocyanates HDI and IPDI.

Therefore, the synthesis had to be re-designed, selecting a different set of diols having simile functionality and aliphatic branches, providing an overall “comb” structure associated with skin adherence and affinity. The use of HDI and IPDI is a necessary compromise with respect to their origin, as no totally bioderived diisocyanates or triisocyanates are available. Moreover, other synthetic routes for obtaining polyurethanes without the use of isocyanates are not yet industrially feasible. Therefore, the strategy object of the present invention in order to reduce the impact of the diisocyanates monomers in the synthesis provides the use of macrodiols with different functionalities, like those typical of polyesters. Finally, if present, the solvent must be replaced by a volatile cosmetic oil of biologic origin or by a non-volatile emollient of natural origin having a suitable compatibility with the polymer itself and the ingredients of the finished product.

Based on the foregoing, the present invention consists in a cosmetic composition for cosmetic products with high naturalness, as defined in claim 1.

The polymers of the present invention are formed by:
a) a polyol polyester with NOI=1, completely made of natural components or of natural origin, in particular of vegetal origin, like glycerol, fatty acids, dicarboxylic acids;
b) optionally with the addition of polyols of a different chemical nature with NOI=1, like hydrogenated dilinoleyl alcohol and hydrogenated castor oil and derivatives;
   in combination with
c) an aliphatic diisocyanate or a triisocyanate, preferably but not necessarily obtained from natural components or of natural origin
   in the presence of:
d) one or more volatile and/or non-volatile emollient cosmetic oils with high NOI (>0.85), having the function of solvent in the synthesis, like e.g. Coco-Caprylate/Caprate, Caprylic/Capric Triglyceride, Tridecane, Undecane, Triheptanoin, Octyldodecanol.

In particular:
a) Polyester polyol with NOI=1

The polyester polyol made of natural components or of natural origin only is made of:
1) glycerol of natural origin;
2) a dicarboxylic acid (or a mixture of dicarboxylic acids) of natural origin;
3) a natural fatty acid (or a mixture of fatty acids) or a fatty acid (or a mixture of fatty acids) of natural origin.

The glycerol of natural origin can be derived from any oil extractible from plants: mainly from rapeseed, sunflower and palm. Glycerol of vegetal origin can be obtained through different processes of the oleochemical industry, very often as a by-product of the production of substances like fatty acids and biodiesel.

Some examples of processes for obtaining glycerol are:
hydrolysis under pressure of glycerides, transesterification, through alcoholysis of glycerides (in the production process of fatty alcohols or biodiesel);
saponification of glycerides with caustic alkali (process for preparing toilet soaps);
synthesis from propylene;
fermentation of simple sugars, through alcoholic fermentation.

The dicarboxylic acids of natural origin comprise, among others, succinic acid, azelaic acid, sebacic acid, dilinoleic acid. Some examples of dicarboxylic acids available on the market for which the vegetal origin is guaranteed are azelaic acid under the commercial name of Matrilox IA001M (Matrica), derived from the transformation of vegetal oils from sustainable cultures like milk thistle (Silybum marianum), typically grown in Sardinia (Italy), succinic acid with commercial name Biosuccinum (Roquette), derived from a “carbon negative” process of fermentation of biomasses, and dilinoleic acid with commercial name Pripol 1009 (Cargill), derived from linseed oil.

E.g., the polyester polyol with azelaic acid suitably exploits said acid as biologic building block obtained from biorefineries that transform biomasses from a non-edible vegetal source (Cynara cardunculus) growing in arid areas not suitable for growing food. The replacement of succinic acid with azelaic acid in the polyester structure allows to obtain a polyurethane with high NOI with similar properties (gloss and viscosity) but provided with a more aliphatic character (9 vs 4 carbon atoms), capable of improving the compatibility with non-polar ingredients. A further enhancement of the hydrophobic features and of the adhesive features of the material can be obtained by using dilinoleic acid, thanks to its structure having longer hydrocarbon chains, very affine to non-polar matrices.

Fatty acids are a basic product of the oleochemical industry. They can be obtained through different industrial processes, starting from different kinds of plants.

On the market there are available saturated and unsaturated fatty acids, with chains of different lengths, of vegetal origin.

The process of hydrogenation of unsaturated fatty acids allows to obtain materials more stable to oxidation and to rancidity, for this reason preferable for cosmetic use.

Fatty acids or mixtures of fatty acids with chains from 4 carbon atoms up to 50 carbon atoms can be used. In particular, in combination with glycerol, one or more organic diacids (4-50 carbon atoms) and one or more organic monoacids (4-50 carbon atoms) can be used.

The building blocks of the polyester polyol are combined in suitable quantities for balancing the complementary mono-, di- and three-functional reactive portions, and polymerized in linear polyesters with a molecular GPC molecular weight higher than 1000 Da and residual hydroxyl functionalities ranging between 50-250 mgKOH/g (preferably 170-190 mgKOH/g), capable of undergoing an addition of diisocyanate and chain extension.

These features allow to obtain polyols with NOI=1, having modulable viscosity and lubricant properties, suitable for producing cosmetic linear or branched polyurethanes, wherein the introduction of the carbamate group provides further sites for hydrogen bonds promoting molecular interactions between polymeric molecules and with the epidermal substrate, maximizing adherence, affinity for the skin and providing marked film-forming properties.
b) Polyols of other chemical nature with NOI=1, like e.g. dimers of fatty alcohols derived through the hydrogenation of dimers of unsaturated fatty acids, and oils derived through the hydrogenation of natural oils containing multiple hydroxy groups, in particular hydrogenated dilinoleyl alcohol and hydrogenated castor oil.

The optional introduction of these polyols has the aim of further modulating the compatibility and the rheologic and structural features of the obtained materials, making them more affine to a wider range of cosmetic oils, and at the same time, the aim of optimizing the structural and rheologic features of said polymers.

Dimers of fatty alcohols are obtained through the hydrogenation of dimers of unsaturated fatty acids, like e.g. oleic acid, linoleic acid, palmitoleic acid, linolenic acid and arachidonic acid. Preferably, hydrogenated dilinoleyl alcohol is used, under the commercial name of Pripol 2030 (Cargill).

Among the oils derived from the hydrogenation of natural oils containing multiple hydroxy groups, hydrogenated castor oil Cutina HR Flakes (BASF) is preferred.
c) Aliphatic diisocyanate or triisocianate, preferably but not necessarily made partially of natural components or of natural origin.

Diisocyanates and triisocyanates can be selected from a group of molecules comprising isophorone diisocyanate, (IPDI), hexamethylene diisocyanate (HDI), Bis(4-isocyanatocyclohexyl) methane (HMDI). In addition to these isocyanates of fossil origin, today on the market there are available isocyanates of a partially natural origin, like e.g. pentamethylene diisocyanate isocyanurate (PDI trimer) with commercial name Desmodur ECO N7300 (Covestro), having the 68% of bio-based carbon and three isocyanate groups, L-lysine diisocyanate (LDI), and a derivative of HDI with partial natural origin, declared with 32% of bio-based carbon under the commercial name of Tolonate X FLO 100 (Vencorex Chemicals).
d) Volatile and non-volatile cosmetic emollient oils with high NOI (>85%), having the function of solvent for the synthesis, like e.g. (non-exhaustive list) Coco-Caprylate/Caprate, Caprylic/Capric Triglyceride, Tridecane, Undecane, Triheptanoin, Octyldodecanol.

The cosmetic oil having the function of solvent in the synthesis is accurately selected with the aim of obtaining a homogeneous dispersion with the polymer, allowing a better introduction of the new polyurethanes in the cosmetic formulas and enhancing the film-forming, long-lasting adherence to skin and gloss properties.

The materials described in this application are the first born of a new family of polyurethanes of natural origin. Conceived in order to improve skin adherence, glossy look, long lasting properties and good sensorial properties, they are also provided with a high Natural Origin Index that can be exploited to formulate products wherein a high naturalness is required.

In particular, the careful combination of the selected materials can lead to obtain linear polyurethanes and branched polyurethanes with the rheologic features of elastic linear or branched components.

The polyurethanes were successfully synthetized, as in the following EXAMPLES.

### EXAMPLE 1

### Synthesis of a Linear Polyurethane Derived from Succinic Acid (NOI=0.86)

| | | % (W/W) |
|---|---|---|
| PHASE A | Coco-Caprylate/Caprate | 50 |
| | Polyester polyol from succinic acid | 20 |
| | Hydrogenated dilinoleyl alcohol | 16 |
| | Zinc catalyst | 0.02 |
| | IPDI | 13 |
| PHASE B | Alcohol | 0.98 |

Coco-Caprylate/Caprate was chosen as cosmetic oil in which the polyurethane of this example was synthetized. A polyester polyol from succinic acid and hydrogenated dilinoleyl alcohol are dissolved in the solvent in the presence of a zinc catalyst (e.g. Zinc-Octanoate) and IPDI. The mixture is heated to 90° C. and is reacted up to complete conversion of the isocyanate to carbamate groups, in about 4 h. A small aliquot of alcohol (e.g. ethanol) is added to stop polymerization. If present, non-reacted reagents are removed under vacuum before cooling and discharging the product.

### EXAMPLE 2

### Synthesis of a Branched Polyurethane Derived from Azelaic Acid (NOI=0.92)

| | | % (W/W) |
|---|---|---|
| PHASE A | Caprylic/Capric Triglyceride | 50 |
| | Polyester polyol from azelaic acid | 25 |
| | Zinc catalyst | 0.02 |
| | HDI | 7 |
| | PDI trimer | 3 |
| PHASE B | Alcohol | 0.98 |

Caprylic/Capric Triglyceride was chosen as cosmetic oil in which the polyurethane of this example was synthetized. A polyester polyol from azelaic acid is dissolved in the solvent in the presence of a zinc catalyst (e.g. Zinc-Octanoate) and HDI and PDI trimer. The mixture is heated to 100° C. and is reacted up to complete conversion of the isocyanate to carbamate groups, in about 3 h. A small aliquot of alcohol (e.g. ethanol) is added to stop polymerization. If present, non-reacted reagents are removed under vacuum before cooling and discharging the product.

### EXAMPLE 3

### Synthesis of a Branched Polyurethane Derived from Dilinoleic Acid (NOI=0.90)

| | | % (W/W) |
|---|---|---|
| PHASE A | Caprylic/Capric Triglyceride | 50 |
| | Polyester polyol from dilinoleic acid | 33 |
| | Hydrogenated castor oil | 6 |
| | Zinc catalyst | 0.02 |
| | IPDI | 10 |
| PHASE B | Alcohol | 0.98 |

Caprylic/Capric Triglyceride was chosen as cosmetic oil in which the polyurethane of this example was synthetized. A polyester polyol from dilinoleic acid and hydrogenated castor oil are dissolved in the solvent in the presence of a zinc catalyst (e.g. Zinc-Octanoate) and IPDI trimer. The mixture is heated to 80° C. and is reacted up to complete conversion of the isocyanate to carbamate groups, in about 8 h. A small aliquot of alcohol (e.g. ethanol) is added to stop polymerization. If present, non-reacted reagents are removed under vacuum before cooling and discharging the product.

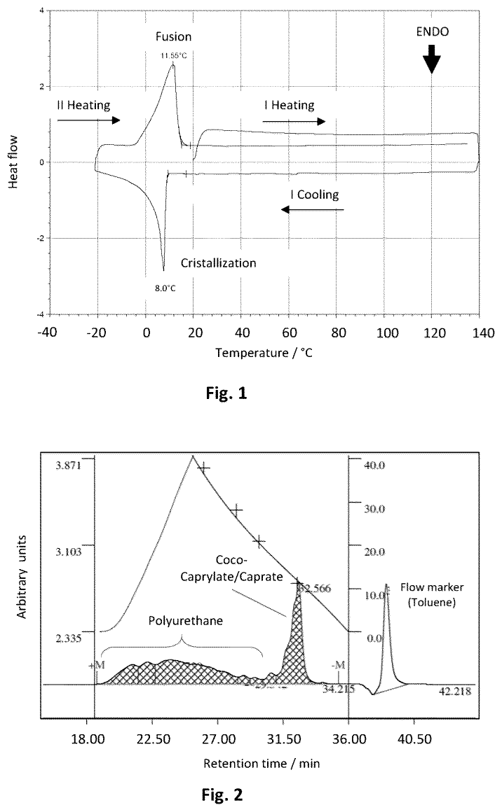

The physical-chemical characterization (ATR-FTIR, DSC, SEC-GPC) of the synthetized polymers was performed, in order to determine their structure, thermal behaviour and molecular weight. See the charts shown in FIGS. 1-3, wherein:

FIG. 1 shows the DSC chart of the linear polyurethane of EXAMPLE 1, showing that the material undergoes a crystallization and fusion associated to the solvent;

FIG. 2 shows the GPC chromatogram of the linear polyurethane of EXAMPLE 1, which shows a molecular weight distribution curve of the material ranging several orders of magnitude (1000 kDa<Mw<1 kDa), with an elevated dispersity and average Mw of about 60 kDa;

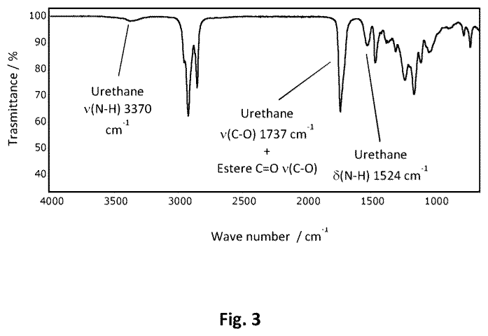

FIG. 3 shows the FT-IR chart of the linear polyurethane of EXAMPLE 1, wherein the disappearance of the NCO signal (absence of the peak at 2226 cm-1) in the IR spectrum of the reaction mixture and the appearance of peaks associated with urethane group show the conversion of isocyanates and the formation of the expected polyurethane.

The new polyurethanes with high NOI according to the present invention were compared to polyurethanes of petrochemical origin based on alkyl tartrate, as shown in the Table hereunder.

| | Silicone | Gloss | | | |
|---|---|---|---|---|---|
| | content | (GU at | | | |
| INGREDIENTS | % | 20°) | Water contact angle (°) | | N.O.I. |
| Linear polyurethane EXAMPLE 1 | 0 | 105 | 78.5 | 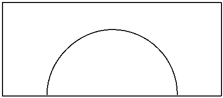 | 0.86 |
| Branched polyurethane EXAMPLE 2 | 0 | 78 | 78.0 | 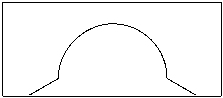 | 0.92 |
| Branched polyurethane EXAMPLE 3 | 0 | 73 | 75.5 | 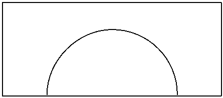 | 0.90 |
| Polyurethane-102 | 2 | 71 | 106 | 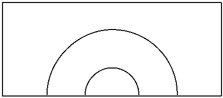 | 0 |
| HDI/Di-C12-14 Alkyl tartrate/ Hydrogenated Dilinoleyl Alcohol Copolimer | 0 | 65 | 96 | 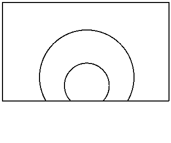 | 0 |
| IPDI/Di-C12-13 Alkyl tartrate/ Bis- Hydroxyethoxypropyl Dimethicone Copolimer | 2 | 62 | 96 | 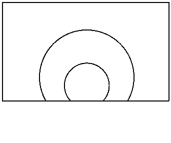 | 0 |
| Bis- Hydroxyethoxypropyl Dimethicone/IPDI Copolimer Ethyl carbamate | 89 | 67 | 123 | 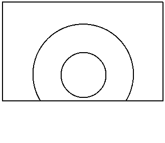 | 0 |

Gloss measurements were performed using a three-angle glossmeter Elcometer 407. Films were prepared on cardboard using a spiral bar as applicator. Gloss was measured after complete drying of the film, when a volatile oil was present. For glossy surfaces, the accepted values are measured at 20°.

The gloss value of 105 GU for the polyurethane of EXAMPLE 1, the value of 78 GU for EXAMPLE 2 and the value of 73 GU for EXAMPLE 3 are higher than the value of HDI/Di-C12-14 Alkyl tartrate/Hydrogenated Dilinoleyl Alcohol Copolimer, showing the cosmetic potential for the development of a glossy finish.

The films so obtained are characterized by non-transfer capacity: in fact, another objective to be tackled is the resistance to water and oils for conferring water-proof and food-proof properties to the cosmetic products containing said polyurethanes.

These features can be detected by using the contact angle with water on thin film layers in order to compare the superficial energy of polymers and to evaluate them consequently. The polyurethanes based on alkyl tartrate have contact angles with water in the interval ranging 90°-100°, associated with overall hydrophobic surfaces with a good resistance to oil. To make a comparison, the Bis-Hydroxyethoxypropyl Dimethicone/IPDI Copolimer Ethyl Carbamate, a silicone-based polyurethane, has a contact angle wider than 120°, associated to the presence of polydimethylsiloxane functions, and is a gold standard concerning film-forming properties for providing water-proof and food-proof properties to the cosmetic products comprising it. In this respect, the new polyurethanes are completely immiscible with water, which is an index of water-proof properties, but at the same time the contact angle of 78.5° for the polyurethane of EXAMPLE 1, the value of 78.0° for EXAMPLE 2 and the value of 75.5° GU for EXAMPLE 3 are index of a higher polarity, which is advantageous for a better adherence to skin, because adherence forces are mediated by polar interactions.

The compatibility with common cosmetic ingredients was studied, and the new materials were used as basic components for innovative make-up formulations.

The expected cosmetic features of the polyurethanes with high NOI, object of the present invention, where verified in different cosmetic formulas, listed hereunder.

In the compact powder eyeshadow of EXAMPLE 4 with finish frost at 2.5% by weight of the overall formula, the new polymer of EXAMPLE 1 was used to successfully improve the adherence and the long-lasting properties of the cosmetic film, contributing at the same time to the shiny aspect of the pearly pigments, without dampening the gloss of pearls, effectively replacing the structuring components of petrochemical origin (Hydrogenated Styrene/Isoprene Copolymer).

On the other hand, in the cosmetic linear or branched lip product of EXAMPLE 5 the new polymers of EXAMPLE 1 and of EXAMPLE 2 were used as agents promoting gloss. The lip liquid provides a uniform, comfortable and flexible film, with a deep chromatic impact and high gloss.

In the lip gloss of EXAMPLE 6, the presence of the branched polyurethane (EXAMPLE 3) allows to provide marked film-forming properties associated with a pleasant texture in application, capable of remaining comfortable and glossy over time.

### EXAMPLE 4

Cosmetic Formula of a Compact Powder Eyeshadow. Percentage of Naturalness NOC=97%

| | % (W/W) |
|---|---|
| Skin conditioning agents | 2 |
| Chelating agents | 0.2 |
| Viscosifying agents | 0.2 |
| Emollient | 14 |
| Skin conditioning agent | 6.6 |
| EXAMPLE 1 sample | 2.5 |
| Preservatives | 1 |
| Colorants | 73.5 |

The product is characterized by a strong intensity of the colour, very good adherence to skin and long-lasting properties over time.

### EXAMPLE 5

Cosmetic Formula of a Lip Liquid. Percentage of Naturalness NOC=80%

| | % (W/W) |
|---|---|
| Skin conditioning agent | 5.8 |
| Emollient | 7.0 |
| Anti-oxidant | 0.1 |
| Emollient | 12.0 |
| Skin conditioning agent | 36.0 |
| EXAMPLE 1 sample | 3 |
| EXAMPLE 2 sample | 2 |
| Viscosifying agents | 3.3 |
| Emollient | 10.3 |
| Colorants | 16.2 |
| Bulking agents | 4.0 |
| Flavouring agents | 0.3 |

The product is characterized by high gloss, long-lasting, water-proof properties and high colour rendering.

### EXAMPLE 6

Cosmetic Formula of a Lip Gloss. Percentage of Naturalness 90%

| | % (W/W) |
|---|---|
| Skin conditioning agent | 5.8 |
| Emollient | 7.0 |
| Anti-oxidant | 0.1 |
| Emollient | 12.0 |
| Skin conditioning agent | 36.0 |
| EXAMPLE 1 sample | 3 |
| EXAMPLE 2 sample | 2 |
| Viscosifying agents | 3.3 |
| Emollient | 10.3 |
| Colorants | 16.2 |
| Bulking agents | 4.0 |
| Flavouring agents | 0.3 |

The product is characterized by high gloss, and long-lasting properties over time.

1-12. (canceled)

13. Cosmetic composition with high naturalness suitable for forming film on skin for cosmetic products, characterized in that the composition comprises at least one linear or branched polyurethane with Natural Origin Index (NOI) higher than 0.85, comprising at least:
a polyol polyester with NOI=1, totally formed by natural or nature-derived components; and
an aliphatic diisocyanate and/or triisocyanate,
said composition further comprising at least one cosmetic oil with function of solvent for the synthesis of polyurethane, wherein said components of polyol polyester with NOI=1 are synthetized starting from glycerol, one or more organic diacids (4-50 carbon atoms) and one or more organic monoacids (4-50 carbon atoms).

14. Cosmetic composition according to claim 13, wherein said linear or branched polyurethane is glossy.

15. Cosmetic composition according to claim 14, wherein said linear or branched polyurethane is provided with a gloss expressed in Gloss Units equal or higher than 70.

16. Cosmetic composition according to claim 13, wherein said linear or branched polyurethane is provided of long-lasting, water-proof and food-proof properties.

17. Cosmetic composition according to claim 13, wherein said polyol polyester with NOI=1 consists of:
glycerol of vegetal origin obtained from any oil extractable from plants, in particular from rapeseed, sunflower, palm;
dicarboxylic acid or mixture of dicarboxylic acids of natural origin, in particular succinic acid, azelaic acid, sebacic acid, dilinoleic acid;
fatty acid or mixture of fatty acids of vegetal origin.

18. Cosmetic composition according to claim 13, wherein said aliphatic diisocyanate and/or triisocyanate consists of natural or nature-derived components.

19. Cosmetic composition according to claim 13, wherein said aliphatic diisocyanate and/or triisocyanate is selected from isophorone diisocyanate, (IPDI), hexamethylene diisocyanate (HDI), Bis(4-isocyanatocyclohexyl) methane (HMDI), pentamethylene diisocyanate isocyanurate (PDI trimer), L-lysine diisocyanate (LDI), and a derivative of HDI with 32% of bio-based carbon.

20. Cosmetic composition according to claim 13, wherein said cosmetic oil is an emollient cosmetic oil, in particular Coco-Caprylate/Caprate, Caprylic/Capric Triglyceride, Tridecane, Undecane, Triheptanoin, Octyldodecanol.

21. Cosmetic composition according to claim 13, wherein said linear or branched polyurethane further comprises polyols of other chemical nature with NOI=1, said polyols being selected from hydrogenated dilinoleyl alcohol, hydrogenated castor oil, dimers of fatty alcohols obtained through the hydrogenation of dimers of unsaturated fatty acids, and oils derived through the hydrogenation of natural oils containing multiple hydroxy groups.

22. Cosmetic composition according to claim 20, wherein the reaction between said polyol polyester, diisocyanate and/or triisocyanate and optionally polyols of other chemical nature with NOI=1 occurs in presence of said cosmetic oil with function of solvent for the synthesis of polyurethane.

23. Cosmetic composition according to claim 13, wherein having a formulation with Natural Origin Content (NOC)>80%.
